(19) United States
(12) Patent Application Publication
(10) Pub. No.: US 2024/0216449 A1
(43) Pub. Date: Jul. 4, 2024

(54) STRAINS OF PROBIOTIC BACTERIA FOR USE IN A PREVENTIVE METHOD OF TREATMENT OR IN AN ADJUVANT METHOD OF TREATMENT OF VIRAL RESPIRATORY INFECTIONS

(71) Applicant: PROBIOTICAL S.P.A., Novara (IT)

(72) Inventors: Vera MOGNA, Novara (IT); Angela AMORUSO, Novara (IT); Marco PANE, Novara (IT)

(21) Appl. No.: 18/560,643

(22) PCT Filed: Jun. 13, 2022
(86) PCT No.: PCT/IB2022/055436

§ 371 Date: Nov. 13, 2023

(30) Foreign Application Priority Data
Jun. 11, 2021 (IT) 102021000015398

(51) Int. Cl. A61K 35/747; A61K 35/00; A61K 35/745; A61P 31/14

(52) U.S. Cl. CPC A61K 35/747; A61K 35/745; A61P 31/14; A61K 2035/115

(57) ABSTRACT

The present invention relates to specific and selected strains of bacteria and compositions thereof for use in the treatment of viral respiratory tract infections, preferably coronavirus viral infections, such as severe acute respiratory syndrome by coronavirus (COVID-19).

### TECHNICAL FIELD

The present invention relates to specific and selected strains of bacteria and compositions thereof for use in the preventive treatment/method of preventing or inhibiting or reducing viral respiratory tract infections, preferably coronavirus viral infections, such as severe acute respiratory syndrome by coronavirus (COVID-19), by receptor inhibition of transmission of a virus, for example, by ACE2 receptor inhibition.

### BACKGROUND

Viral respiratory tract infections, as their name implies, are infectious diseases caused by bacteria or viruses that affect the organs of the upper and/or lower respiratory tract (nose, trachea, bronchi and lungs).

Among the respiratory diseases caused by viruses, infections linked to RNA viruses are more notable than those caused by DNA viruses. Five families of RNA viruses that cause upper and lower respiratory tract infections have been identified: Orthomyxoviridae (comprising influenza viruses A, B, C or D), Paramyxoviridae and Pneumoviridae (comprising parainfluenza virus, respiratory syncytial virus and human metapneumovirus), Picornaviridae (rhinoviruses A, B, and C and respiratory enteroviruses A, B, C and D are included in this family), and Coronaviridae (which includes human coronavirus, severe acute respiratory syndrome coronavirus and middle eastern respiratory syndrome coronavirus).

The Coronaviridae family is a very important RNA virus family that has caused respiratory infections such as the epidemic caused by SARS-CoV (severe acute respiratory syndrome coronavirus), MERS-CoV (middle-eastern respiratory syndrome coronavirus) and the SARS-CoV-2 pandemic, (severe acute respiratory syndrome coronavirus 2), which started in 2020 and is still ongoing.

Coronaviruses are known to predominantly target the human respiratory system, as previously observed with SARS-CoV and MERS-CoV. Phylogenetic and computational genomic analyses suggest that in order to enter host cells, SARS-CoV-2 shares the same human cellular receptor with SARS-CoV (angiotensin-converting enzyme 2, ACE2). Specifically, when the organism is infected with a virus belonging to the Coronaviridae family, such as SARS-CoV-2, the viral Spike protein binds the ACE2 receptor (a homologue of the angiotensin-converting enzyme (ACE) present on the organism's cell surface.

The Spike protein (S protein, SPIKE) is a type I membrane glycoprotein that facilitates the attachment of a virus belonging to the Coronaviridae family to cell receptors and its subsequent entry into the host cell. This protein consists of a short intracellular tail, a transmembrane anchor and a large ectodomain. The ectodomain comprises two subunits: S1 (N-terminal surface unit) and S2 (C-terminal transmembrane unit). The distal S1 subunit comprises the receptor-binding domain (RBD), which is responsible for binding to the host cell receptor, and contributes to the stabilisation of the pre-fusion state of the membrane-anchored S2 subunit, which contains the functional elements required for membrane fusion.

ACE2 is a homologue of the angiotensin-converting enzyme (ACE) and is mainly expressed on type 2 pneumocytes in the lungs and weakly expressed on the surface of epithelial cells of the oral and nasal mucosa and nasopharynx, indicating that the lungs are the primary target of a virus belonging to the Coronaviridae family, such as SARS-CoV-2. The formation of the binding of the viral spike protein with the ACE2 receptor is thus the basis for the initiation of viral infection of the respiratory tract.

Probably the most effective way to control this type of infectious disease is vaccination, however, this is a type of preventive remedy that serves to immunise the population against a viral infection, such as the current SARS-CoV-2 infection. However, vaccines are not always available for all types of viral respiratory tract infections, and these can be difficult to obtain or very expensive.

Once the infection has been contracted, there are certain treatments that can be administered, such as antiviral drugs, said treatments being known in the art, however, these treatments are not always as effective as desirable, and they can also have significant side effects.

Therefore, there is a need for new products for the preventive treatment or inhibition or reduction of respiratory tract infections of a viral nature.

Following intensive research and development activity carried out by the Applicant, the present invention addresses and solves the technical problems set forth in the present description by providing specific and selected strains of bacteria (e.g., strains of probiotic bacteria or derivatives thereof, parabiotics or postbiotics), mixtures and compositions comprising at least one of said strains of bacteria (probiotics or parabiotics or postbiotics thereof) for use in a preventive method of treatment or inhibiting, or reducing said respiratory tract infections having a viral nature.

In particular, the applicant surprisingly found that the bacterial strains described herein and compositions comprising them can be effectively used in the preventive treatment of viral respiratory tract infections.

Furthermore, the Applicant has surprisingly found that the bacterial strains described herein and compositions comprising them can be effectively used in the treatment of viral respiratory tract infections at an early stage of infection, as the formation of the bond between the viral Spike protein and the ACE2 receptor is the basis for the initiation of viral respiratory tract infection.

Moreover, the Applicant has surprisingly found that the strains of bacteria described herein and the compositions comprising them can be effectively used in combination with an antiviral treatment.

Without wishing to be bound by specific scientific theories, it is hypothesised that this positive effect may also derive from the fact that probiotic bacteria may have effects on the modulation of the gut microbiota and immune responses, particularly those occurring in the upper/lower respiratory tract, and may therefore improve the host response to respiratory viral infections in particular.

The specific strains of bacteria (e.g. probiotic bacterial strains or derivatives thereof) and their compositions according to the present invention are advantageously capable of significantly inhibiting and/or reducing the binding between ACE2 and the RBD (receptor-binding domain) of the Spike protein.

The bacterial strains of the present invention, mixtures and compositions of the present invention comprising said bacterial strains, exhibit a high safety profile, can be used by all categories of subjects, are easy to prepare and cost-effective.

These purposes, and others that will become clear from the detailed description that follows, are achieved by the bacteria strains, mixtures and compositions of the present invention due to the technical features present in the description and in the appended claims.

### SUMMARY OF THE INVENTION

It is an object of the present invention a strain of bacteria for use in a treatment method of preventing or inhibiting, or reducing, a viral infection of the respiratory system in a subject in need thereof, and associated diseases or symptoms, wherein said strain of bacteria is selected from the group consisting of, or alternatively consisting of:
(i) a strain of bacteria belonging to the species Lactobacillus brevis, wherein said strain of bacteria is selected from the group comprising or, alternatively, consisting of a strain of bacteria identified as Lactobacillus brevis LBR01 (DSM 23034);
(ii) a strain of bacteria belonging to the species Lactobacillus crispatus, wherein said strain of bacteria is selected from the group comprising or, alternatively, consisting of Lactobacillus crispatus LCR01 (DSM 24619), Lactobacillus crispatus LCR04 (DSM 33487), and mixtures thereof;
(iii) a strain of bacteria belonging to the species Limosilactobacillus fermentum (formerly Lactobacillus fermentum), wherein said strain of bacteria is selected from the group comprising or, alternatively, consisting of Limosilactobacillus fermentum (formerly Lactobacillus fermentum) LF10 (DSM 19187), Limosilactobacillus fermentum (formerly Lactobacillus fermentum) LF15 (DSM 26955), and mixtures thereof;
(iv) a strain of bacteria belonging to the species Lactobacillus gasseri, wherein said strain of bacteria is selected from the group comprising or, alternatively, consisting of a strain of bacteria identified as Lactobacillus gasseri LGS01(DSM 18299);
(v) a strain of bacteria belonging to the species Lactiplantibacillus plantarum (formerly Lactobacillus plantarum), wherein said strain of bacteria is selected from the group comprising or, alternatively, consisting of Lactiplantibacillus plantarum (formerly Lactobacillus plantarum) LP01 (LMG P-21021), Lactiplantibacillus plantarum (formerly Lactobacillus plantarum) LP02 (LMG P-21020), Lactiplantibacillus plantarum (formerly Lactobacillus plantarum) LP09 (DSM 25710), Lactiplantibacillus plantarum (formerly Lactobacillus plantarum) LP14 (DSM 33401) and mixtures thereof;
(vi) a strain of bacteria belonging to the species Limosilactobacillus reuteri (formerly Lactobacillus reuteri), wherein said strain of bacteria is selected from the group comprising or, alternatively, consisting of Limosilactobacillus reuteri (formerly Lactobacillus reuteri) LRE02 (DSM 23878), Limosilactobacillus reuteri (formerly Lactobacillus reuteri) LRE03 (DSM 23879), Limosilactobacillus reuteri (formerly Lactobacillus reuteri) LRE11 (DSM 33827), and mixtures thereof;
(vii) a strain of bacteria belonging to the species Lactobacillus salivarius sub. salivarius, wherein said strain of bacteria is selected from the group comprising or, alternatively, consisting of a strain of bacteria identified as Lactobacillus salivarius sub. salivarius LS03 (DSM 22776);
(viii) a strain of bacteria belonging to the species Bifidobacterium adolescentis, wherein said strain of bacteria is selected from the group comprising or, alternatively, consisting of Bifidobacterium adolescentis BA02 (EI-15) (DSM 18351), and mixtures thereof;
(ix) a strain of bacteria belonging to the species Bifidobacterium bifidum, wherein said strain of bacteria is selected from the group comprising or, alternatively, consisting of Bifidobacterium bifidum BB01 (DSM 22892), Bifidobacterium bifidum BB10 (DSM 33678), and mixtures thereof;
(x) a strain of bacteria belonging to the species Bifidobacterium breve, wherein said strain of bacteria is selected from the group comprising or, alternatively, consisting of Bifidobacterium breve BR03 (DSM 16604), Bifidobacterium breve B632 (DSM 24706), and mixtures thereof;
(xi) a strain of bacteria belonging to the species Bifidobacterium infantis, wherein said strain of bacteria is selected from the group comprising or, alternatively, consisting of Bifidobacterium infantis BI02 (DSM 24687);
(xii) a strain of bacteria belonging to the species Bifidobacterium lactis, wherein said strain of bacteria is selected from the group comprising or, alternatively, consisting of Bifidobacterium lactis BS01 (LMG P-21384);
(xiii) a strain of bacteria belonging to the species Bifidobacterium longum, wherein said strain of bacteria is selected from the group comprising or, alternatively, consisting of Bifidobacterium longum BL03 (DSM 16603), Bifidobacterium longum BL04 (DSM 23233), Bifidobacterium longum DLBL07 (DSM 25669), Bifidobacterium longum DLBL08 (DSM 25670), Bifidobacterium longum DLBL 10 (DSM 25672), Bifidobacterium longum DLBL 11 (DSM 25673), and mixtures thereof.

It is also an object of the present invention, a mixture for use in a treatment method of preventing or inhibiting, or reducing a viral infection of the respiratory system in a subject in need thereof, and associated diseases or symptoms, having the characteristics as defined in the appended claims.

It is also an object of the present invention, a composition for use in a treatment method of preventing, or inhibiting, a viral infection of the respiratory system in a subject, and associated diseases or symptoms, comprising:
a strain of bacteria according to the appended claims, or a mixture of strains according to the appended claims; and
a food-grade or pharmaceutically acceptable additive or excipient.

### Definitions

In the context of the present invention, the term “respiratory tract”, “respiratory apparatus” means the set of organs and tissues deputed to the breathing process. The main anatomical elements of the respiratory apparatus are: the nose with its cavities, the mouth, the pharynx, the nasopharynx, the larynx, the trachea, the bronchi, the bronchioles, the lungs and the diaphragm and intercostal breathing muscles.

In the context of the present invention, the term ‘respiratory tract infections’ refers to infectious diseases affecting the organs of the respiratory apparatus (nose, trachea, bronchi and lungs).

In the context of the present invention, the term “coronavirus” means a virus of the family Coronaviridae, subfamily: Coronavirinae, genus: Betacoronavirus, species: severe acute respiratory syndrome coronavirus or severe acute respiratory syndrome-related coronavirus (in short, SARSr-CoV or SARS-coronavirus or coronavirus), selected from the following strains: (I) severe acute respiratory syndrome coronavirus (SARS-CoV) (isolated and first identified in 2002), (II) severe acute respiratory syndrome coronavirus-2 (SARS-CoV-2) (isolated and first identified in 2019), and (III) severe acute respiratory syndrome coronavirus-like (SARS-CoV-like).

### DETAILED DESCRIPTION OF THE INVENTION

It is an object of the present invention a strain of bacteria for use in a treatment method of preventing or inhibiting, or reducing, a viral infection of the respiratory apparatus in a subject in need thereof, and associated diseases or symptoms, wherein said strain of bacteria is selected from the group comprising, or alternatively consisting of:
(i) a strain of bacteria belonging to the species Lactobacillus brevis, preferably a strain of bacteria identified as Lactobacillus brevis LBR01 (DSM 23034);
(ii) a strain of bacteria belonging to the species Lactobacillus crispatus, preferably selected from the group consisting of Lactobacillus crispatus LCR01 (DSM 24619), Lactobacillus crispatus LCR04 (DSM 33487), and mixtures thereof;
(iii) a strain of bacteria belonging to the species Limosilactobacillus fermentum (formerly Lactobacillus fermentum), preferably selected from the group consisting of Limosilactobacillus fermentum (formerly Lactobacillus fermentum) LF10 (DSM 19187), Limosilactobacillus fermentum (formerly Lactobacillus fermentum) LF15 (DSM 26955), and mixtures thereof;
(iv) a strain of bacteria belonging to the species Lactobacillus gasseri, preferably a strain of bacteria identified as Lactobacillus gasseri LGS01(DSM 18299);
(v) a strain of bacteria belonging to the species Lactiplantibacillus plantarum (formerly Lactobacillus plantarum), preferably selected from the group consisting of Lactiplantibacillus plantarum (formerly Lactobacillus plantarum) LP01 (LMG P-21021) Lactiplantibacillus plantarum (formerly Lactobacillus plantarum) LP02 (LMG P-21020), Lactiplantibacillus plantarum (formerly Lactobacillus plantarum) LP09 (DSM 25710), Lactiplantibacillus plantarum (formerly Lactobacillus plantarum) LP14 (DSM 33401) and mixtures thereof;
(vi) a strain of bacteria belonging to the species Limosilactobacillus reuteri (formerly Lactobacillus reuteri), preferably selected from the group consisting of Limosilactobacillus reuteri (formerly Lactobacillus reuteri) LRE02 (DSM 23878) Limosilactobacillus reuteri (formerly Lactobacillus reuteri) LRE03 (DSM 23879), Limosilactobacillus reuteri (formerly Lactobacillus reuteri) LRE11 (DSM 33827), and mixtures thereof;
(vii) a strain of bacteria belonging to the species Lactobacillus salivarius sub. salivarius, preferably a strain of bacteria identified as Lactobacillus salivarius sub. salivarius LS03 (DSM 22776);
(viii) a strain of bacteria belonging to the species Bifidobacterium adolescentis, preferably selected from the group consisting of Bifidobacterium adolescentis BA02 (EI-15) (DSM 18351), Bifidobacterium adolescentis BA05 (EI-18) (DSM 18352), and mixtures thereof;
(ix) a strain of bacteria belonging to the species Bifidobacterium bifidum, preferably selected from the group consisting of Bifidobacterium bifidum BB01 (DSM 22892), Bifidobacterium bifidum BB10 (DSM 33678), and mixtures thereof;
(x) a strain of bacteria belonging to the species Bifidobacterium breve, preferably selected from the group consisting of Bifidobacterium breve BR03 (DSM 16604), Bifidobacterium breve B632 (DSM 24706), and mixtures thereof;
(xi) a strain of bacteria belonging to the species Bifidobacterium infantis, preferably identified as Bifidobacterium infantis BI02 (DSM 24687);
(xii) a strain of bacteria belonging to the species Bifidobacterium lactis, preferably identified as Bifidobacterium lactis BS01 (LMG P-21384);
(xiii) a strain of bacteria belonging to the species Bifidobacterium longum, preferably selected from the group consisting of Bifidobacterium longum BL03 (DSM 16603), Bifidobacterium longum BL04 (DSM 23233) Bifidobacterium longum DLBL07 (DSM 25669), Bifidobacterium longum DLBL08 (DSM 25670), Bifidobacterium longum DLBL 10 (DSM 25672), Bifidobacterium longum DLBL 11 (DSM 25673), and mixtures thereof.

All strains of bacteria cited in the present invention were deposited in accordance with the provisions of the Budapest Treaty. The Depositor of the strains of bacteria described and/or claimed in the present patent application and the owner thereof hereby express their consent to make all said strains available for the whole duration of the patent.

The species nomenclature of strains of bacteria belonging to the genus Lactobacillus was amended following the reclassification of said genus, as reported in the article by Zheng et al., nt. J. Syst. Evol. Microbiol., 70(4):2782-2858 (2020). In the context of the present invention, the species nomenclature adopted after said reclassification is adopted, the name according to the old classification being indicated in brackets.

Below are the deposit specifications of the bacteria strains used in this context: Lactobacillus brevis LBR01, deposited, in accordance with the provisions of the Budapest Treaty, at the Deutsche Sammlung von Mikroorganismen und Zellkulturen GmbH (DSMZ) under deposit number DSM 23034 on 13 Oct. 2009 by Probiotical S.p.A.

Lactobacillus crispatus LCR01 deposited, in accordance with the provisions of the Budapest Treaty, at the Deutsche Sammlung von Mikroorganismen und Zellkulturen GmbH (DSMZ) under deposit number DSM 24619 on 2 Mar. 2011 by Probiotical S.p.A.

Lactobacillus crispatus LCR04 deposited, in accordance with the provisions of the Budapest Treaty, at the Deutsche Sammlung von Mikroorganismen und Zellkulturen GmbH (DSMZ) under deposit number (DSM 33487) on 2 Apr. 2020 by Probiotical S.p.A.

Limosilactobacillus fermentum (formerly Lactobacillus fermentum) LF10 deposited, in accordance with the provisions of the Budapest Treaty, at the Deutsche Sammlung von Mikroorganismen und Zellkulturen GmbH (DSMZ) under deposit number (DSM 19187) on 20 Mar. 2007 by Anidral S.r.L. (now Probiotical S.p.A.).

Limosilactobacillus fermentum (formerly Lactobacillus fermentum) LF15 deposited, in accordance with the provisions of the Budapest Treaty, at the Deutsche Sammlung von Mikroorganismen und Zellkulturen GmbH (DSMZ) under deposit number (DSM 26955) on 1 Mar. 2013 by Probiotical S.p.A.

Lactobacillus gasseri LGS01 deposited, in accordance with the provisions of the Budapest Treaty, at the Deutsche Sammlung von Mikroorganismen und Zellkulturen GmbH (DSMZ) under deposit number DSM 18299; on 24 May 2006 by Anidral Srl (now Probiotical S.p.A.).

Lactiplantibacillus plantarum (formerly Lactobacillus plantarum) LP01 deposited, in accordance with the provisions of the Treaty of Budapest, at the Belgian Coordinated Collections of Microorganisms (BCCM) Laboratorium voor Microbiologie—Bacteriënverzameling (LMG) under deposit number LMG P-21021; on 16 Oct. 2001, by Mofin Srl.

Lactiplantibacillus plantarum (formerly Lactobacillus plantarum) LP02 deposited, in accordance with the provisions of the Treaty of Budapest, at the Belgian Coordinated Collections of Microorganisms (BCCM) Laboratorium voor Microbiologie—Bacteriënverzameling (LMG) under deposit number LMG P-21020; on 16 Oct. 2001 by Mofin Srl.

Lactiplantibacillus plantarum (formerly Lactobacillus plantarum) LP09 deposited, in accordance with the provisions of the Budapest Treaty, at the Deutsche Sammlung von Mikroorganismen und Zellkulturen GmbH (DSMZ) under deposit number DSM 25710; on 24 Feb. 2012, by Probiotical S.p.A.

Lactiplantibacillus plantarum (formerly Lactobacillus plantarum) LP14 deposited, in accordance with the provisions of the Budapest Treaty, at the Deutsche Sammlung von Mikroorganismen und Zellkulturen GmbH (DSMZ) under deposit number DSM 33401; on 16 Jan. 2020, by Probiotical S. p. A.

Limosilactobacillus reuteri (formerly Lactobacillus reuteri) LRE02 deposited, in accordance with the provisions of the Budapest Treaty, at the Deutsche Sammlung von Mikroorganismen und Zellkulturen GmbH (DSMZ) under deposit number DSM 23878; on 5 Aug. 2010, by Probiotical S. p. A.

Limosilactobacillus reuteri (formerly Lactobacillus reuteri) LRE03 deposited, in accordance with the provisions of the Budapest Treaty, at the Deutsche Sammlung von Mikroorganismen und Zellkulturen GmbH (DSMZ) under deposit number DSM 23879, on 5 Aug. 2010, by Probiotical S. p. A.

Limosilactobacillus reuteri (formerly Lactobacillus reuteri) LRE11 deposited, in accordance with the provisions of the Budapest Treaty, at the Deutsche Sammlung von Mikroorganismen und Zellkulturen GmbH (DSMZ) under deposit number DSM 33827, on 15 Feb. 2021, by Probiotical S. p. A.

Lactobacillus salivarius sub. salivarius LS03 deposited, in accordance with the provisions of the Budapest Treaty, at the Deutsche Sammlung von Mikroorganismen und Zellkulturen GmbH (DSMZ) under deposit number DSM 22776, on 23 Jul. 2009, by Probiotical S. p. A.

Bifidobacterium adolescentis BA02 (EI-15) deposited, in accordance with the provisions of the Budapest Treaty, at the Deutsche Sammlung von Mikroorganismen und Zellkulturen GmbH (DSMZ) under deposit number DSM 18351, on 15 Jun. 2006, by Anidral Srl (now Probiotical S.p.A.).

Bifidobacterium adolescentis BA05 (EI-18) deposited, in accordance with the provisions of the Budapest Treaty, at the Deutsche Sammlung von Mikroorganismen und Zellkulturen GmbH (DSMZ) under deposit number DSM 18352, on 15 Jun. 2006, by Anidral Srl (now Probiotical S.p.A.).

Bifidobacterium bifidum BB01 deposited, in accordance with the provisions of the Budapest Treaty, at the Deutsche Sammlung von Mikroorganismen und Zellkulturen GmbH (DSMZ) under deposit number DSM 19818, on 30 Oct. 2007, by Anidral Srl (now Probiotical S.p.A.).

Bifidobacterium bifidum BB10 deposited, in accordance with the provisions of the Budapest Treaty, at the Deutsche Sammlung von Mikroorganismen und Zellkulturen GmbH (DSMZ) under deposit number DSM 33678, on 22 Oct. 2020, by Probiotical S.p.A.

Bifidobacterium breve BR03 deposited, in accordance with the provisions of the Budapest Treaty, at the Deutsche Sammlung von Mikroorganismen und Zellkulturen GmbH (DSMZ) under deposit number DSM 16604, on 20 Jul. 2004, by Anidral Srl (now Probiotical S.p.A.).

Bifidobacterium breve B632 deposited, in accordance with the provisions of the Budapest Treaty, at the Deutsche Sammlung von Mikroorganismen und Zellkulturen GmbH (DSMZ) under deposit number DSM 24706, on 7 Apr. 2011, by Probiotical S. p. A.

Bifidobacterium infantis B102 deposited, in accordance with the provisions of the Budapest Treaty, at the Deutsche Sammlung von Mikroorganismen und Zellkulturen GmbH (DSMZ) under deposit number DSM 24687, on 29 Mar. 2011, by Probiotical S.p.A.

Bifidobacterium lactis BS01 deposited, in accordance with the provisions of the Budapest Treaty, at the Belgian Coordinated Collections of Microorganisms (BCCM) Laboratorium voor Microbiologie—Bacteriënverzameling (LMG) under deposit number LMG P-21384, on 31 Jan. 2002, by Anidral srl (now Probiotical S.p.A.).

Bifidobacterium longum BL03 deposited, in accordance with the provisions of the Budapest Treaty, at the Deutsche Sammlung von Mikroorganismen und Zellkulturen GmbH (DSMZ) under deposit number DSM 16603, on 20 Jul. 2004 by Anidral srl (now Probiotical S.p.A.).

Bifidobacterium longum BL04 deposited, in accordance with the provisions of the Budapest Treaty, at the Deutsche Sammlung von Mikroorganismen und Zellkulturen GmbH (DSMZ) under deposit number DSM 23233, on 12 Jan. 2010, by Probiotical S. p. A.

Bifidobacterium longum DLBL07 deposited, in accordance with the provisions of the Budapest Treaty, at the Deutsche Sammlung von Mikroorganismen und Zellkulturen GmbH (DSMZ) under deposit number DSM 25669, on 16 Feb. 2012, by Probiotical S. p. A.

Bifidobacterium longum DLBL08 deposited, in accordance with the provisions of the Budapest Treaty, at the Deutsche Sammlung von Mikroorganismen und Zellkulturen GmbH (DSMZ) under deposit number DSM 25670, on 16 Feb. 2012, by Probiotical S. p. A.

Bifidobacterium longum DLBL 10 deposited, in accordance with the provisions of the Budapest Treaty, at the Deutsche Sammlung von Mikroorganismen und Zellkulturen GmbH (DSMZ) under deposit number DSM 25672, on 16 Feb. 2012, by Probiotical S. p. A.

Bifidobacterium longum DLBL 11 deposited, in accordance with the provisions of the Budapest Treaty, at the Deutsche Sammlung von Mikroorganismen und Zellkulturen GmbH (DSMZ) under deposit number DSM 25673, on 16 Feb. 2012, by Probiotical S. p. A.

The strains used in the present context, or mixtures thereof, may be strains of viable probiotic bacteria or derivatives thereof, such as parabiotics or postbiotics. Preferably, the strains of bacteria used in the present context are strains of probiotic/viable/living bacteria, or cell extracts or supernatants of said strains of bacteria.

In the context of the present invention, the term ‘probiotic’ means a live, viable strain of bacteria that, when administered in adequate amounts, provides a health benefit to the host (or official FAO and WHO 2002 definition).

In the context of the present invention, the term ‘derivative’ of a viable probiotic strain of bacteria means a paraprobiotic or a postbiotic or any other derivative of the strain of bacteria having the capacity to provide a benefit to the organism to which they are administered (in analogy to the viable strain of bacteria from which they are derived).

In the context of the present invention, the term “paraprobiotics” means bacterial cells (intact or non-intact) that are non-viable (i.e., not capable of replication) or cell extracts, e.g., crude cell extracts, which, when administered in adequate amounts, provide a health benefit to the host (in analogy to the viable bacterial strain from which they are derived). Examples of paraprobiotics are bacterial strains inactivated by heat (e.g. tindalised bacterial strains), sonication (ultrasound), gammation (gamma rays), or lysates of bacterial strains or cell extracts of bacterial strains.

In the context of the present invention, the term ‘postbiotics’ means any substance released or produced through the metabolic and catabolic activity of the viable probiotic bacterial strain, wherein said postbiotics, when administered in adequate amounts, provide a health benefit to the host (in analogy to the viable bacterial strain from which they are derived). Examples of postbiotics are exopolysaccharides, wall fractions, metabolites or metabolic bioproducts, or the supernatant obtained from bacterial strains.

The term ‘subject(s)’ in the context of the present invention refers to mammals (animal and human), preferably human subjects.

The term ‘therapeutically effective amount refers to the amount of mixture or compound or formulation that elicits the biological or medicinal response in a tissue, system or subject that is sought and defined by an expert in the field.

The bacterial strains described in this context act through a preventive antiviral action and restoration of the gut microbiome.

In addition, the strains of bacteria described herein and mixtures and compositions comprising them are for use in the curative treatment of viral infections of the respiratory tract at an early stage of infection, preferably at a stage between day 1 and day 14 after virus contraction, preferably at a stage between day 1 and day 10 after virus contraction.

Furthermore, the applicant surprisingly found that the bacterial strains described herein and the compositions comprising them can be effectively used in combination with an antiviral treatment.

Moreover, the bacterial strains described herein are readily available, easy to administer (oral administration), safe and cheap compared to antiviral drugs and immunomodulators.

The strains, mixtures and compositions as described in the present invention are for use in a preventive treatment method, i.e. it allows limiting/reducing the risk of contracting a viral infection by preventing it or reducing the likelihood of contracting it.

The strains, mixtures and compositions as described in the present invention are for use in a curative treatment method, intended as a method of treatment that at an early stage after virus contraction, preferably within 14 after contraction, allows limiting/reducing the spread of the viral infection by reducing/alleviating the initial symptoms.

Preferably, the mixtures described herein are for use in a preventive treatment method, or inhibition, or reduction, of a viral infection of the respiratory tract by a coronavirus, preferably SARS-CoV-2 virus, in a subject in need thereof, and associated diseases or symptoms.

Preferably, the compositions described herein are for use in a preventive treatment method, or inhibition, or reduction, of a viral infection of the respiratory tract by a coronavirus, preferably SARS-CoV-2 virus, in a subject in need thereof, and associated diseases or symptoms.

The strains, mixtures and compositions as described in the present invention are for use in an adjuvant treatment method that can be administered in concomitance with antiviral therapy to enhance its effects and to help strengthen the immune system and the health of the microbiota of the subject suffering from a viral respiratory tract infection.

It is an object of the present invention a strain of bacteria for use in a treatment method of preventing, or inhibiting, or reducing a viral infection of the respiratory system in a subject in need thereof, and associated diseases or symptoms, wherein said strain of bacteria is selected from the group comprising or alternatively, consisting of:
Lactobacillus brevis LBR01 (DSM 23034);
Lactobacillus crispatus LCR01 (DSM 24619);
Lactobacillus crispatus LCR04 (DSM 33487);
Limosilactobacillus fermentum (formerly Lactobacillus fermentum) LF10 (DSM19187);
Limosilactobacillus fermentum (formerly Lactobacillus fermentum) LF15 (DSM 26955);
Lactobacillus gasseri LGS01 (DSM 18299);
Lactiplantibacillus plantarum (formerly Lactobacillus plantarum) LP01 (LMG P-21021);
Lactiplantibacillus plantarum (formerly Lactobacillus plantarum) LP02 (LMG P-21020);
Lactiplantibacillus plantarum (formerly Lactobacillus plantarum) LP09 (DSM 25710);
Lactiplantibacillus plantarum (formerly Lactobacillus plantarum) LP14 (DSM 33401);
Limosilactobacillus reuteri (formerly Lactobacillus reuteri) LRE02 (DSM 23878);
Limosilactobacillus reuteri (formerly Lactobacillus reuteri) LRE03 (DSM 23879);
Limosilactobacillus reuteri (formerly Lactobacillus reuteri) LRE11 (DSM 33827);
Lactobacillus salivarius sub. salivarius LS03 (DSM 22776);
Bifidobacterium adolescentis BA02 (EI-15) (DSM 18351);
Bifidobacterium bifidum BB01 (DSM 22892);
Bifidobacterium bifidum BB10 (DSM 33678);
Bifidobacterium breve BR03 (DSM 16604);
Bifidobacterium breve B632 (DSM 24706);
Bifidobacterium infantis BI02 (DSM 24687);
Bifidobacterium lactis BS01 (LMG P-21384);
Bifidobacterium longum BL03 (DSM 16603);
Bifidobacterium longum BL04 (DSM23233);
Bifidobacterium longum DLBL07 (DSM 25669);
Bifidobacterium longum DLBL08 (DSM 25670);
Bifidobacterium longum DLBL 10 (DSM 25672);
Bifidobacterium longum DLBL 11 (DSM 25673),
and mixture thereof.

Preferably, said strain of bacteria for use in a preventive treatment method/for preventing or inhibiting or reducing a viral respiratory tract infection in a subject in need thereof, and associated diseases or symptoms, wherein said strain of bacteria is selected from the group comprising or, alternatively, consisting of: Lactobacillus crispatus LCR01 (DSM 24619), Lactobacillus crispatus LCR04 (DSM 33487), Limosilactobacillus fermentum (formerly Lactobacillus fermentum) LF15 (DSM 26955), Lactiplantibacillus plantarum (formerly Lactobacillus plantarum) LP01 (LMG P-21021), Lactiplantibacillus plantarum (formerly Lactobacillus plantarum) LP02 (LMG P-21020), L. salivarius sub. Salivarius LS03 (DSM 22776), Bifidobacterium bifidum BB10 (DSM 33678), Lactiplantibacillus plantarum (formerly Lactobacillus plantarum) LP09 (DSM 25710), Lactiplantibacillus plantarum (Lactobacillus plantarum) LP14 (DSM 33401), Bifidobacterium longum BL03 (DSM 16603), Lactobacillus gasseri LGS01 (DSM 18299), and mixtures thereof.

Preferably, said strain of bacteria is selected from the group comprising or, alternatively, consisting of: Lactobacillus crispatus LCR01 (DSM 24619), Lactobacillus crispatus LCR04 (DSM 33487), Limosilactobacillus fermentum (formerly Lactobacillus fermentum) LF15 (DSM 26955), Lactiplantibacillus plantarum (formerly Lactobacillus plantarum) LP01 (LMG P-21021), Lactiplantibacillus plantarum (formerly Lactobacillus plantarum) LP02 (LMG P-21020), Lactobacillus salivarius sub. salivarius LS03 (DSM 22776), Bifidobacterium bifidum BB10 (DSM 33678), and mixtures thereof.

Alternatively, said strain of bacteria is selected from the group comprising or, alternatively, consisting of: Lactobacillus crispatus LCR01 (DSM 24619), Lactobacillus crispatus LCR04 (DSM 33487), Lactiplantibacillus plantarum (formerly Lactobacillus plantarum) LP01 (LMG P-21021), Lactiplantibacillus plantarum (formerly Lactobacillus plantarum) LP02 (LMG P-21020), Lactobacillus salivarius sub. salivarius LS03 (DSM 22776), Lactiplantibacillus plantarum (formerly Lactobacillus plantarum) LP09 (DSM 25710), Lactiplantibacillus plantarum (Lactobacillus plantarum) LP14 (DSM 33401), Bifidobacterium longum BL03 (DSM 16603), Lactobacillus gasseri LGS01(DSM 18299), and mixtures thereof.

Advantageously, the strains of bacteria described herein may be viable/probiotic strains, or in the form of cell extracts or supernatant obtainable from said strains of bacteria.

The present invention further relates to a supernatant obtainable from a strain of bacteria according to any one of the embodiments described herein, preferably, wherein said supernatant is obtainable from a strain of bacteria selected from the group comprising or, alternatively, consisting of: Lactobacillus crispatus LCR01 (DSM 24619), Lactobacillus crispatus LCR04 (DSM 33487), Lactiplantibacillus plantarum (formerly Lactobacillus plantarum) LP01 (LMG P-21021), Lactiplantibacillus plantarum (formerly Lactobacillus plantarum) LP02 (LMG P-21020), Lactobacillus salivarius sub. salivarius LS03 (DSM 22776), Lactiplantibacillus plantarum (formerly Lactobacillus plantarum) LP09 (DSM 25710), Lactiplantibacillus plantarum (formerly Lactobacillus plantarum) LP14 (DSM 33401), Bifidobacterium longum BL03 (DSM 16603), Lactobacillus gasseri LGS01 (DSM 18299), and mixtures thereof.

In an embodiment form, said strain of bacteria is in the form of a live/probiotic strain, and is preferably selected from the group comprising or, alternatively, consisting of: Lactobacillus crispatus LCR01 (DSM 24619), Lactobacillus crispatus LCR04 (DSM 33487), Limosilactobacillus fermentum (formerly Lactobacillus fermentum) LF15 (DSM 26955), Lactiplantibacillus plantarum (formerly Lactobacillus plantarum) LP01 (LMG P-21021), Lactiplantibacillus plantarum (formerly Lactobacillus plantarum) LP02 (LMG P-21020), Lactobacillus salivarius sub. salivarius LS03 (DSM 22776), Bifidobacterium bifidum BB10 (DSM 33678), and mixtures thereof.

In an embodiment, the strain of bacteria for use according to the present invention is selected from the group comprising or, alternatively, consisting of:
(ii) a strain of bacteria belonging to the species Lactobacillus crispatus, selected from the group comprising or alternatively, consisting of Lactobacillus crispatus LCR01 (DSM 24619), Lactobacillus crispatus LCR04 (DSM 33487), and mixtures thereof;
(iii) a strain of bacteria belonging to the species Limosilactobacillus fermentum (formerly Lactobacillus fermentum), selected from the group comprising or, alternatively, consisting of Limosilactobacillus fermentum (formerly Lactobacillus fermentum) LF15 (DSM 26955), and mixtures thereof;
(v) a strain of bacteria belonging to the species Lactiplantibacillus plantarum (formerly Lactobacillus plantarum), selected from the group comprising or, alternatively, consisting of Lactobacillus plantarum LP01 (LMG P-21021), Lactiplantibacillus plantarum (formerly Lactobacillus plantarum) LP02 (LMG P-21020), and mixtures thereof;
(vii) a strain of bacteria belonging to the species Lactobacillus salivarius sub. salivarius, selected from the group comprising or, alternatively, consisting of a strain of bacteria identified as Lactobacillus salivarius sub. salivarius LS03 (DSM 22776);
(ix) a strain of bacteria belonging to the species Bifidobacterium bifidum selected from the group comprising or, alternatively, consisting of Bifidobacterium bifidum BB01 (DSM 22892), Bifidobacterium bifidum BB10 (DSM 33678) and mixtures thereof;

It is also an object of the present invention a mixture for use in a method of treatment of a viral infection of the respiratory apparatus in a subject in need thereof, and associated diseases or symptoms, comprising or, alternatively, consisting of at least one strain of bacteria described herein.

The mixture of the present invention preferably comprises at least two strains of bacteria, or at least three strains, or at least 4 strains, or at least 5 strains selected from the group comprising or, alternatively, consisting of:
Lactobacillus brevis LBR01 (DSM 23034);
Lactobacillus crispatus LCR01 (DSM 24619);
Lactobacillus crispatus LCR04 (DSM 33487);
Limosilactobacillus fermentum (formerly Lactobacillus fermentum) LF10 (DSM 19187);
Limosilactobacillus fermentum (formerly Lactobacillus fermentum) LF15 (DSM 26955);
Lactobacillus gasseri LGS01(DSM 18299);
Lactiplantibacillus plantarum (formerly Lactobacillus plantarum) LP01 (LMG P-21021);
Lactiplantibacillus plantarum (formerly Lactobacillus plantarum) LP02 (LMG P-21020);
Lactiplantibacillus plantarum (formerly Lactobacillus plantarum) LP09 (DSM 25710);
Lactiplantibacillus plantarum (formerly Lactobacillus plantarum) LP14 (DSM 33401);
Limosilactobacillus reuteri (formerly Lactobacillus reuteri) LRE02 (DSM 23878);
Limosilactobacillus reuteri (formerly Lactobacillus reuteri) LRE03 (DSM 23879);
Limosilactobacillus reuteri (formerly Lactobacillus reuteri) LRE11 (DSM 33827);
Lactobacillus salivarius sub. salivarius LS03 (DSM 22776);
Bifidobacterium adolescentis BA02 (EI-15) (DSM 18351);
Bifidobacterium bifidum BB01 (DSM 22892);
Bifidobacterium bifidum BB10 (DSM 33678);
Bifidobacterium breve BR03 (DSM 16604);
Bifidobacterium breve B632 (DSM 24706);
Bifidobacterium infantis BI02 (DSM 24687);
Bifidobacterium lactis BS01 (LMG P-21384);
Bifidobacterium longum BL03 (DSM 16603);
Bifidobacterium longum BL04 (DSM23233);
Bifidobacterium longum DLBL07 (DSM 25669);
Bifidobacterium longum DLBL08 (DSM 25670);
Bifidobacterium longum DLBL 10 (DSM 25672);
Bifidobacterium longum DLBL 11 (DSM 25673).

Preferably, said mixture comprises at least two strains selected from the group consisting of Lactobacillus crispatus LCR01 (DSM 24619), Lactobacillus crispatus LCR04 (DSM 33487), Limosilactobacillus fermentum (formerly Lactobacillus fermentum) LF15 (DSM 26955), Lactiplantibacillus plantarum (formerly Lactobacillus plantarum) LP01 (LMG P-21021), Lactiplantibacillus plantarum (formerly Lactobacillus plantarum) LP02 (LMG P-21020), Lactobacillus salivarius sub. salivarius LS03 (DSM 22776), Bifidobacterium bifidum BB10 (DSM 33678), Lactobacillus plantarum LP09 (DSM 25710), Lactiplantibacillus plantarum (formerly Lactobacillus plantarum) LP14 (DSM 33401), Bifidobacterium longum BL03 (DSM 16603), Lactobacillus gasseri LGS01 (DSM 18299).

In an embodiment, said mixture comprises Lactobacillus crispatus LCR01 (DSM 24619), Lactobacillus crispatus LCR04 (DSM 33487), Limosilactobacillus fermentum (formerly Lactobacillus fermentum) LF15 (DSM 26955), Lactiplantibacillus plantarum (formerly Lactobacillus plantarum) LP01 (LMG P-21021), Lactiplantibacillus plantarum (formerly Lactobacillus plantarum) LP02 (LMG P-21020), Lactobacillus salivarius sub. salivarius LS03 (DSM 22776), Bifidobacterium bifidum BB10 (DSM 33678), (formerly Lactiplantibacillus plantarum) Lactobacillus plantarum LP09 (DSM 25710), Lactiplantibacillus plantarum (formerly Lactobacillus plantarum) LP14 (DSM 33401), Bifidobacterium longum BL03 (DSM 16603), Lactobacillus gasseri LGS01(DSM 18299).

The mixture described in this context may also include at least one prebiotic ingredient or prebiotic fibre; preferably selected from the group comprising or, alternatively, consisting of: inulin, fructo-oligosaccharide (FOS), galacto-oligosaccharide (GOS), guar gum and their mixtures, preferably inulin.

It is also an object of the present invention a composition comprising at least one strain of bacteria as described herein, or a mixture of strains of bacteria as described herein, and an additive and/or excipient of pharmaceutically or food acceptable grade. Said composition may advantageously be used in a preventive treatment method or inhibition or reduction of a viral infection of the respiratory apparatus in a subject in need thereof, and associated pathologies or symptoms.

In the context of the present invention, ‘composition’ means a composition that may be a pharmaceutical composition (or Live Biotherapeutic Products), a medical device composition (e.g. Medical Device Regulation (EU) 2017/745 (MDR)), a food supplement and/or a food for special medical purposes (FSMP), or a composition for cosmetic use.

Said at least one strain of bacteria may be present in the mixture M or composition of the present invention, with respect to a dosage unit of said composition or mixture M of the present invention, in a concentration in the range from 10×10⁶ CFU to 10×10¹² CFU (e.g. 10×10⁷ CFU or 10×10⁸ CFU or 10×10⁹ CFU or 10×10¹⁰ CFU), preferably about from 1×10⁹ CFU to 10×10⁹ CFU, (CFU: Colony Forming Unit).

When more than one strain of bacteria of the present invention are included in the mixture M or composition of the present invention, said two, three, four, five or six strains of bacteria may be in a ratio expressed in CFU of about 1:1, or about 1:1:1, or about 1:1:1:1, or about 1:1:1:1:1, or about 1:1:1:1:1:1 (CFU: Colony Forming Unit).

In order to assess the number of live bacteria in the compositions or mixtures M of the present invention, said compositions or mixtures M can be analysed e.g. by flow cytometry, to determine the AFU value, and/or by plate count method, to determine the CFU value.

The above dosage units can be administered to the subject in need thereof one, two, three or four times a day, preferably two.

Said compositions of the present invention, comprising at least one strain of bacteria of the present invention or comprising a mixture of strains of bacteria, according to any one of the described embodiments), may be formulated for oral use (gastroenteric or sublingual), for nasal inhalation (e.g., sprays or drops), for oral inhalation (e.g., sprays, dry powders for inhalation), or for topical use (e.g., dermal, rectal, vaginal or ophthalmic). Preferably, the compositions or mixtures of the present invention are for oral use.

Said composition of the present invention may be formulated for oral use in a solid form, e.g., selected from: tablets, chewable tablets, orosoluble tablets, capsules, granules, flakes, powder, soluble powder or granules (e.g., packaged in sachets), orosoluble powder or granules (e.g., packaged in orosoluble sticks); or, alternatively, in a liquid form, e.g., selected from: solutions, suspensions, emulsions, syrups, e.g. packaged in drinkable vials, dispensable liquid in a spray form; or, alternatively, in semi-liquid form, e.g., selected from: soft-gel, gel or cream; preferably the composition or mixture of the present invention is for oral use in a solid form, more preferably in soluble or water-soluble powder form (e.g. in water or water-based liquids).

Said at least one additive and/or excipient of pharmaceutical or food grade included in the composition of the invention, together with the mixture M comprising at least one strain of probiotic bacteria or derivatives thereof of the present invention, is a substance without therapeutic activity suitable for pharmaceutical or food grade use selected from auxiliary substances known to the person skilled in the art such as, e.g., diluents, solvents, solubilizers, thickeners, sweeteners, flavourings, colouring agents, lubricants, surfactants, antimicrobials, antioxidants, preservatives, pH stabilising buffers and mixtures thereof.

### Experimental Part

### Methods

The strains of bacteria according to the present invention used in the following exemplary experimental part of the invention are listed in Table 1.

All the bacterial strains cited in the present invention were deposited in accordance with the provisions of the Budapest Treaty. The Depositor (Probiotical SpA) of the strains of bacteria described and/or claimed in this patent application and the owner (Probiotical SpA), of the same, hereby express their consent to make all said strains available for the whole duration of the patent.

TABLE 1
Strains of Bacteria.

| | | |
|---|---|---|
| Lactobacillus brevis | LBR01 | DSM 23034 |
| Lactobacillus crispatus | LCR01 | DSM 24619 |
| Lactobacillus crispatus | LCR04 | DSM 33487 |
| Limosilactobacillus fermentum | LF10 | DSM 19187 |
| Limosilactobacillus fermentum | LF15 | DSM 26955 |
| Lactobacillus gasseri | LGS01 | DSM 18299 |
| Lactiplantibacillus plantarum | LP01 | LMG P-21021 |
| Lactiplantibacillus plantarum | LP02 | LMG P-21020 |
| Lactiplantibacillus plantarum | LP09 | DSM 25710 |
| Lactiplantibacillus plantarum | LP14 | DSM 33401 |
| Limosilactobacillus reuteri | LRE02 | DSM 23878 |
| Limosilactobacillus reuteri | LRE03 | DSM 23879 |
| Limosilactobacillus reuteri | LRE11 | DSM 33827 |
| L. salivarius sub. salivarius | LS03 | DSM 22776 |
| Bifidobacterium adolescentis | BA02 | DSM 18351 |
| | (EI-15) | |
| Bifidobacterium adolescentis | BA05 | DSM 18352 |
| | (EI-18) | |
| Bifidobacterium bifidum | BB01 | DSM 22892 |
| Bifidobacterium bifidum | BB10 | DSM 33678 |
| Bifidobacterium breve | BR03 | DSM 16604 |
| Bifidobacterium breve | B632 | DSM 24706 |
| Bifidobacterium infantis | BI02 | DSM 24687 |
| Bifidobacterium lactis | BS01 | LMG P-21384 |
| Bifidobacterium longum | BL03 | DSM 16603 |
| Bifidobacterium longum | BL04 | DSM 23233 |
| Bifidobacterium longum | DLBL07 | DSM 25669 |
| Bifidobacterium longum | DLBL08 | DSM 25670 |
| MIX Bifidobacterium longum | DLBL07\ | DSM 25669 |
| | 10\11 | DSM 25672 |
| | | DSM 25673 |

Prior to use, the probiotic strains were stored in 20% glycerol at −80° C.

They were thawed and cultured overnight at 37° C. in de Man, Rogosa and Sharpe broth (MRS).

As Bifidobacterium species are more sensitive to O₂ concentration than Lactobacillus species, MRS was enriched with 1% by volume of a cysteine solution to thaw it. The next morning, the fresh cultures were used for the experimental part described in this context.

Efficacy in the treatment of respiratory tract infections was tested with live bacteria, but also with strains of bacteria after thawing and freezing treatment (cell extract), and supernatant was also tested. The results showed that not only live strains of bacteria, but also the supernatant and the cell extract were effective for the treatment of viral respiratory tract infections.

Preparation of Supernatant and Extract from Bacteria Strains

Three to four passages after thawing, the bacterial strains were used for the experiments. They were counted by flow cytometry, verifying that all bacterial strains showed an amount of active fluorescent units (AFU) greater than 1×10⁹ ml.

To prepare the supernatant, 5 ml was taken from the bacterial culture and subjected to centrifugation for 15 min at 4000 rpm. At the end of centrifugation, the supernatant was recovered and stored in a refrigerator at 4° C. until use.

To obtain the cell extracts from 5 ml of bacterial culture, a thawing and freezing treatment was carried out. The heat treatment consisted of a heating phase in microwaves, 20 seconds, power 700 W, followed by a freezing phase at −80° C. The procedure was repeated a total of four times. At the end of the last cycle, the cell extracts thus obtained were counted by flow cytometry to verify the absence of viable cells in the samples. At this point, each cell extract was read on the spectrophotometer to reach an optical density (OD) close to 1 and resuspended in Dulbecco's buffered saline solution (DPBS) for use.

### Inhibitor Screening Assay

In order to evaluate the effect of the bacterial strains according to the present invention in the preventive treatment of viral respiratory tract infections, the ability of the bacterial strains listed in Table 1 to inhibit the binding of viruses belonging to the Coronaviridae family was evaluated. The results for the SARS-CoV-2 virus are shown below, but similar results were obtained for the other viruses tested.

The ‘SARS-CoV-2 Inhibitor Screening Assay’ from AdipoGen Life Sciences (AG-48B-0001-K101) was used to test the ability to inhibit the binding of SARS-CoV-2 viruses to the human receptor. This inhibitor screening assay is based on a colorimetric sandwich E.L.I.S.A. Specifically, this kit measures the binding of the receptor binding domain (RBD) of the SARS-CoV-2 spike protein to its human angiotensin-converting enzyme 2 (ACE2) receptor.

The investigation was carried out five times and three different conditions (in triplicate) were tested: live bacterial strains, cell extracts, and supernatant.

Each set of experiments included the following experimental points: negative controls (wherein the binding between spike protein and ACE2 is verified), positive inhibition controls (wherein the ability of the anti-ACE2 antibody to prevent the interaction between ACE2 and the RBD of the used spike protein is verified), experimental points wherein the media used for bacterial growth was verified to have no inhibiting ability between RBD and ACE2, and finally the experimental points to verify the inhibiting efficacy of the RBD-ACE2 binding of viable strains, their supernatants and their cell extracts.

### Plate Preparation

A 96-well plate was coated by adding 100 μl/well of antigen, which in this case was RBD of the SARS-CoV-2 spike protein (SPIKE) (1 μg/ml). Briefly, the lyophilised SPIKE was reconstituted with 100 μl deionised water to obtain 0.1 mg/ml; the reconstituted SPIKE was diluted to the working concentration of 1 μg/ml in DPBS. Then, the plate was covered with plastic film and left overnight at 4° C. in the refrigerator.

The next day, the remaining liquid from the coated wells was removed by vacuuming and inverting the plate and blotting it against clean absorbent paper. Next, 200 μl of Blocking Buffer was added to block the SPIKE protein on the plate and incubated for 2 hours at room temperature. Then, the coated wells were aspirated and washed three times with 300 μl of Wash Buffer 1×. After the last wash, any residual Wash Buffer 1× was removed by inverting the plate and blotting it against clean absorbent paper.

At this point, the three wells corresponding to the negative control were treated with 200 μl of biotinylated ACE2 (0.5 μg/ml). Briefly, the lyophilised ACE2 was reconstituted with 100 μl deionised water to obtain a concentration of 0.1 mg/ml; it was then diluted and mixed with ELISA buffer 1× to obtain a final concentration of 0.5 μg/ml. In the second negative control well, 100 μl of MRS broth was also added; while in the third negative control well, 100 μl of DPBS was also added. On the other hand, the three wells corresponding to the positive control were treated with 200 μl of anti-ACE2 antibody (human), mAb (blocking) (AC384) (INHIB, 1 μg/ml). INHIB is an antibody that inhibits the ACE2 receptor and prevents its interaction with SPIKE and the enzyme peroxidase: thus, no enzymatic reaction should occur in the positive control. To obtain the final concentration of 1 μg/ml, 1 mg/ml INHIB was diluted with ACE2 (0.5 μg/ml). As was done for the negative control, 100 μl of MRS broth was also added to the second well of the positive control, while 100 μl of DPBS was also included in the third well. The respective remaining wells were stimulated with 100 μl/well of bacterial strains (100×10⁶ AFU), supernatants and cell extracts according to the present invention (previously prepared) and 100 μl/well of biotinylated ACE2 (0.5 μg/ml) was added.

Finally, the plate was covered with plastic film and incubated overnight at 37° C. in a humidified atmosphere of 5% CO₂.

At the beginning of the third and last day, washes were performed by adding 300 μl of Wash Buffer 1× and the procedure was repeated three times. After the last wash, any residual Wash Buffer 1× was removed by inverting the plate and blotting it against clean absorbent paper.

After adding 100 μl of streptavidin-labelled horseradish peroxidase (HRP), the plate was covered with plastic film and incubated for 2 hours at room temperature. HRP is able to bind to the biotin of ACE2, due to the fact that streptavidin, to which it is conjugated, has 4 binding sites with high affinity for biotin.

At the end of the incubation, the three washes and removal of the Wash Buffer 1× were repeated as described before.

Then, 100 μl/well of K-Blue Aqueous tetramethylbenzidine (TMB) was added and incubated at RT for 5 minutes. TMB is a 100% solvent-free substrate; it contains 3,3′,5,5′ tetramethylbenzidine and hydrogen peroxide and develops a deep blue colour in the presence of conjugated and labelled peroxidase.

Finally, 50 μl/well of stop solution (H₂ SO₄, 2M) was added to inhibit the enzymatic activity of peroxidase. The addition of sulphuric acid not only inhibits the development of colour, but also converts the blue oxidation product of the TMB into a yellow derivative, which has a significantly higher molar absorbance at 450 nm. The absorbance of this coloured solution was quantified by spectrophotometer (λ=450 nm).

### Statistical Analysis

The results were expressed as mean±standard deviation of the average of at least 3 different independent experiments. Statistical analysis was performed using Student's t-test. Values of p<0.05 were considered significant.

### Results

The bacterial strains tested modulate the binding of RBD to the human ACE2 receptor.

As previously reported, in order to identify and characterise the effect of bacterial strains on the inhibition of RBD binding of SARS-CoV-2 spike proteins to the human ACE2 receptor, the ‘SARS-CoV-2 Inhibitor Screening Assay’ was used. Three different conditions were tested: probiotic bacteria strains (live), their supernatant, normalised by cell number, and cell extracts.

The plate was coated with the RBD domain of the spike protein. The negative control wells were treated with ACE2, which can bind to the RBD; in contrast, the positive control wells were treated with ACE2 and anti-ACE2 human monoclonal antibody to measure the ability of the anti-ACE2 antibody to prevent the interaction between ACE2 and the RBD. The remaining wells were treated with ACE2 and the different bacterial strains.

The final results are expressed as the percentage of inhibition of binding between ACE2 and RBD, compared to the negative control (−).

For the positive control, an inhibition percentage of 51.73±11.28% was obtained, compared to 0% for the negative control.

Interestingly, all the bacterial strains tested showed the ability to inhibit the binding of ACE2 and RBD differently, as shown in Table 2.

TABLE 2
inhibition % of probiotic strains, supernatants and cell
extracts vs. − negative control; + positive control.

| | | | AVERAGE | Standard |
|---|---|---|---|---|
| | | | (inhibition | deviation |
| | | | %) | (SD) |
| − | no | | 0 | |
| | stimulus | | | |
| + | receptor + | | 51.73 | 11.28 |
| | inhibitor | | | |
| Lactobacillus | LBR01 | Vital strain | 35.2 | 23.6 |
| brevis | | Cell extract | 27.6 | 12.7 |
| Lactobacillus | LCR01 | Vital strain | 61.0 | 2.0 |
| crispatus | | supernatant | 79.6 | 1.8 |
| | | Cell extract | 20.0 | 5.8 |
| Lactobacillus | LCR04 | Vital strain | 60.9 | 1.6 |
| crispatus | | supernatant | 80.0 | 1.8 |
| | | Cell extract | 21.0 | 6.2 |
| Limosilactobacillus | LF10 | Vital strain | 4.5 | 21.5 |
| fermentum | | supernatant | 41.9 | 15.6 |
| | | Cell extract | 12.1 | 28.7 |
| Limosilactobacillus | LF15 | Vital strain | 53.2 | 13.5 |
| fermentum | | supernatant | 19.0 | 37.9 |
| | | Cell extract | 7.7 | 14.5 |
| Lactobacillus | LGS01 | Vital strain | 47.0 | 10.6 |
| gasseri | | supernatant | 77.8 | 3.9 |
| | | Cell extract | 5.1 | 1.4 |
| Lactiplantibacillus | LP01 | Vital strain | 51.9 | 7.4 |
| plantarum | | supernatant | 78.2 | 2.4 |
| | | Cell extract | 10.4 | 12.0 |
| Lactiplantibacillus | LP02 | Vital strain | 59.6 | 0.5 |
| plantarum | | supernatant | 80.5 | 1.1 |
| | | Cell extract | 7.6 | 6.5 |
| Lactiplantibacillus | LP09 | Vital strain | 38.6 | 15.7 |
| plantarum | | supernatant | 74.0 | 5.1 |
| | | Cell extract | 14.1 | 20.5 |
| Lactiplantibacillus | LP14 | Vital strain | 38.6 | 15.7 |
| plantarum | | supernatant | 74.0 | 5.1 |
| | | Cell extract | 14.1 | 20.5 |
| Limosilactobacillus | LRE02 | Vital strain | 35.8 | 5.7 |
| reuteri | | supernatant | 41.8 | 7.8 |
| | | Cell extract | 28.5 | 7.6 |
| Limosilactobacillus | LRE03 | Vital strain | 4.6 | 18.3 |
| reuteri | | supernatant | 15.5 | 17.2 |
| | | Cell extract | 9.9 | 33.4 |
| Limosilactobacillus | LRE11 | Vital strain | 4.6 | 18.3 |
| reuteri | | supernatant | 15.5 | 17.2 |
| | | Cell extract | 9.9 | 33.4 |
| L. salivarius | LS03 | Vital strain | 49.5 | 3.4 |
| sub. salivarius | | supernatant | 78.5 | 2.9 |
| | | Cell extract | 14.8 | 8.2 |
| Bifidobacterium | BA02 | Vital strain | 5.0 | 42.6 |
| adolescentis | (EI-15) | supernatant | 19.5 | 71.6 |
| | | Cell extract | 25.0 | 12.3 |
| Bifidobacterium | BB01 | Vital strain | 30.9 | 22.0 |
| bifidum | | Cell extract | 23.4 | 15.0 |
| Bifidobacterium | BB10 | Vital strain | 53.4 | 40.0 |
| bifidum | | supernatant | 16.5 | 43.9 |
| | | Cell extract | 30.0 | 18.3 |
| Bifidobacterium | BR03 | Vital strain | 11.6 | 22.3 |
| breve | | supernatant | 16.0 | 49.3 |
| | | Cell extract | 24.6 | 22.4 |
| Bifidobacterium | B632 | Vital strain | 12.0 | 21.0 |
| breve | | supernatant | 27.5 | 20.0 |
| | | Cell extract | 25.0 | 20.0 |
| Bifidobacterium | BI02 | Cell extract | 18.9 | 24.8 |
| infantis | | supernatant | 9.1 | 42.9 |
| Bifidobacterium | BS01 | Cell extract | 19.5 | 18.8 |
| lactis | | | | |
| Bifidobacterium | BL03 | Vital strain | 27.3 | 35.3 |
| longum | | supernatant | 78.6 | 2.7 |
| | | Cell extract | 14.0 | 6.3 |
| Bifidobacterium | BL04 | Vital strain | 46.7 | 11.2 |
| longum | | supernatant | 48.9 | 45.1 |
| | | Cell extract | 22.9 | 13.9 |
| Bifidobacterium | DLBL07 | supernatant | 54.5 | 20.0 |
| longum | | | | |
| Bifidobacterium | DLBL08 | Vital strain | 7.5 | 4.5 |
| longum | | supernatant | 6.8 | 3.0 |
| | | Cell extract | 14.0 | 20.7 |

The human monoclonal antibody used as a positive control is commercially available as Anti-ACE2 (human)(SARS Receptor, Angiotensin-converting Enzyme 2, ACEH; Metalloprotease MPROT15; SARS-Cov-2 Receptor), mAb (AC384), and was purchased from Adipogen International (product code AG-20A-0037). The above results show that all tested bacteria strains were effective. In particular, excellent results were obtained for the bacteria strains LCR01 and LCR04, LF15, LP01 and LP02, LS03 and BB10. These values are very similar to the value obtained for the positive control, demonstrating that these strains of bacteria are able to inhibit the binding between ACE2 and the RBD of the spike protein.

As shown in Table 2, equally effective are the bacterial strains LCR01, LCR04, LGS01, LP01, LP02, LP09 and LP14, LS03 and BL03.

Analysing the results, it can be seen that amany of the supernatants also show a capacity of over 70% to counteract the binding of the ACE2 receptor to RBD (Table 2).

The effect of the bacterial strains according to the present invention, their supernatants and cell extracts on the inhibition of the binding of the receptor binding domain (RBD) of the SARS-CoV-2 spike proteins to the human ACE2 receptor, particularly relevant for virus entry into the host cell, was demonstrated.

Analysing the results, but without wanting to be bound by specific scientific theories, one can speculate that this inhibitory activity could be due to the release of certain metabolic products of probiotics that may inhibit the binding of RBD to the ACE2 receptor.

From what has been demonstrated above, it can be concluded that the bacterial strains described in this context are a valid solution for the treatment of viral airway infections, in particular for the treatment of viral infection symptoms due to COVID-19. This type of therapy can be used alone or in combination with newly developed therapies or even in support of the vaccination plan.

1. A strain of bacteria for use in a treatment method of preventing or in inhibiting, or reducing a viral infection of the respiratory apparatus in a subject in need thereof, and associated diseases or symptoms, wherein said strain of bacteria is selected from the group comprising or, alternatively, consisting of:
(i) a strain of bacteria belonging to the species Lactobacillus brevis, wherein said strain of bacteria is selected from the group comprising or, alternatively, consisting of a strain of bacteria identified as Lactobacillus brevis LBR01 (DSM 23034);
(ii) a strain of bacteria belonging to the species Lactobacillus crispatus, wherein said strain of bacteria is selected from the group comprising or, alternatively, consisting of Lactobacillus crispatus LCR01 (DSM 24619), Lactobacillus crispatus LCR04 (DSM 33487), and mixtures thereof;
(iii) a strain of bacteria belonging to the species Limosilactobacillus fermentum (formerly Lactobacillus fermentum), wherein said strain of bacteria is selected from the group comprising or, alternatively, consisting of Limosilactobacillus fermentum (formerly Lactobacillus fermentum) LF10 (DSM19187), Limosilactobacillus fermentum (formerly Lactobacillus fermentum) LF15 (DSM 26955), and mixtures thereof;
(iv) a strain of bacteria belonging to the species Lactobacillus gasseri, wherein said strain of bacteria is selected from the group comprising or, alternatively, consisting of a strain of bacteria identified as Lactobacillus gasseri LGS01(DSM 18299);
(v) a strain of bacteria belonging to the species Lactiplantibacillus plantarum (formerly Lactobacillus plantarum), wherein said strain of bacteria is selected from the group comprising or, alternatively, consisting of Lactiplantibacillus plantarum (formerly Lactobacillus plantarum) LP01 (LMG P-21021) Lactiplantibacillus plantarum (formerly Lactobacillus plantarum) LP02 (LMG P-21020), Lactiplantibacillus plantarum (formerly Lactobacillus plantarum) LP09 (DSM 25710), Lactiplantibacillus plantarum (formerly Lactobacillus plantarum) LP14 (DSM 33401) and mixtures thereof;
(vi) a strain of bacteria belonging to the species Limosilactobacillus reuteri (formerly Lactobacillus reuteri), wherein said strain of bacteria is selected from the group comprising or, alternatively, consisting of Limosilactobacillus reuteri (formerly Lactobacillus reuteri) LRE02 (DSM 23878), Limosilactobacillus reuteri (formerly Lactobacillus reuteri) LRE03 (DSM 23879), Limosilactobacillus reuteri (formerly Lactobacillus reuteri) LRE11 (DSM 33827), and mixtures thereof;
(vii) a strain of bacteria belonging to the species Lactobacillus salivarius sub. salivarius, wherein said strain of bacteria is selected from the group comprising or, alternatively, consisting of a strain of bacteria identified as Lactobacillus salivarius sub. salivarius LS03 (DSM 22776);
(viii) a strain of bacteria belonging to the species Bifidobacterium adolescentis, wherein said strain of bacteria is selected from the group comprising or, alternatively, consisting of Bifidobacterium adolescentis BA02 (EI-15) (DSM 18351), and mixtures thereof;
(ix) a strain of bacteria belonging to the species Bifidobacterium bifidum wherein said strain of bacteria is selected from the group comprising or, alternatively, consisting of Bifidobacterium bifidum BB01 (DSM 22892), Bifidobacterium bifidum BB10 (DSM 33678), and mixtures thereof;
(x) a strain of bacteria belonging to the species Bifidobacterium breve, wherein said strain of bacteria is selected from the group comprising or, alternatively, consisting of Bifidobacterium breve BR03 (DSM 16604), Bifidobacterium breve B632 (DSM 24706), and mixtures thereof;
(xi) a strain of bacteria belonging to the species Bifidobacterium infantis, wherein said strain of bacteria is selected from the group comprising or, alternatively, consisting of Bifidobacterium infantis B102 (DSM 24687);
(xii) a strain of bacteria belonging to the species Bifidobacterium lactis, wherein said strain of bacteria is selected from the group comprising or, alternatively, consisting of Bifidobacterium lactis BS01 (LMG P-21384);
(xiii) a strain of bacteria belonging to the species Bifidobacterium longum, wherein said strain of bacteria is selected from the group comprising or, alternatively, consisting of Bifidobacterium longum BL03 (DSM 16603), Bifidobacterium longum BL04 (DSM23233), Bifidobacterium longum DLBL07 (DSM 25669), Bifidobacterium longum DLBL08 (DSM 25670), Bifidobacterium longum DLBL 10 (DSM 25672), Bifidobacterium longum DLBL 11 (DSM 25673), and mixtures thereof.

2. The strain of bacteria for use according to claim 1, wherein said strain of bacteria is alive or dead or tindalised, preferably is a viable probiotic strain of bacteria or alternatively is a cell extract or supernatant of said viable strain of bacteria.

3. The strain of bacteria for use according to claim 1, wherein said strain of bacteria is selected from the group consisting of, or alternatively consisting of:
(i) a strain of bacteria belonging to the species Lactobacillus brevis, wherein said strain of bacteria is selected from the group comprising or, alternatively, consisting of a strain of bacteria identified as Lactobacillus brevis LBR01 (DSM 23034);
(ii) a strain of bacteria belonging to the species Lactobacillus crispatus, wherein said strain of bacteria is selected from the group comprising or, alternatively, consisting of Lactobacillus crispatus LCR01 (DSM 24619), Lactobacillus crispatus LCR04 (DSM 33487), and mixtures thereof;
(iii) a strain of bacteria belonging to the species Limosilactobacillus fermentum (formerly Lactobacillus fermentum), wherein said strain of bacteria is selected from the group comprising or, alternatively, consisting of Limosilactobacillus fermentum (formerly Lactobacillus fermentum) LF10 (DSM19187), Limosilactobacillus fermentum (formerly Lactobacillus fermentum) LF15 (DSM 26955), and mixtures thereof;
(iv) a strain of bacteria belonging to the species Lactobacillus gasseri, wherein said strain of bacteria is selected from the group comprising or, alternatively, consisting of a strain of bacteria identified as Lactobacillus gasseri LGS01(DSM 18299);
(v) a strain of bacteria belonging to the species Lactiplantibacillus plantarum (formerly Lactobacillus plantarum), wherein said strain of bacteria is selected from the group comprising or, alternatively, consisting of Lactobacillus plantarum LP01 (LMG P-21021), Lactiplantibacillus plantarum (formerly Lactobacillus plantarum) LP02 (LMG P-21020), Lactiplantibacillus plantarum (formerly Lactobacillus plantarum) LP09 (DSM 25710), Lactiplantibacillus plantarum (formerly Lactobacillus plantarum) LP14 (DSM 33401) and mixtures thereof;
(vii) a strain of bacteria belonging to the species Lactobacillus salivarius sub. salivarius, wherein said strain of bacteria is selected from the group comprising or, alternatively, consisting of a strain of bacteria identified as Lactobacillus salivarius sub. salivarius LS03 (DSM 22776);
(ix) a strain of bacteria belonging to the species Bifidobacterium bifidum wherein said strain of bacteria is selected from the group comprising or, alternatively, consisting of Bifidobacterium bifidum BB01 (DSM 22892), Bifidobacterium bifidum BB10 (DSM 33678), and mixtures thereof;
(xii) a strain of bacteria belonging to the species Bifidobacterium lactis, wherein said strain of bacteria is selected from the group comprising or, alternatively, consisting of Bifidobacterium lactis BS01 (LMG P-21384); and mixtures thereof.

4. The strain of bacteria for use according to claim 1, wherein said strain of bacteria is in the form of a viable/probiotic strain and is, preferably, selected from the group comprising or, alternatively, consisting of: Lactobacillus crispatus LCR01 (DSM 24619), Lactobacillus crispatus LCR04 (DSM 33487), Limosilactobacillus fermentum (formerly Lactobacillus fermentum) LF15 (DSM 26955), Lactiplantibacillus plantarum (formerly Lactobacillus plantarum) LP01 (LMG P-21021), Lactiplantibacillus plantarum (formerly Lactobacillus plantarum) LP02 (LMG P-21020), Lactobacillus salivarius sub. salivarius LS03 (DSM 22776), Bifidobacterium bifidum BB10 (DSM 33678), and mixtures thereof.

5. A supernatant obtainable from a strain of bacteria according to claim 1, preferably, wherein said supernatant is obtainable from a strain of bacteria selected from the group comprising or, alternatively, consisting of: Lactobacillus crispatus LCR01 (DSM 24619), Lactobacillus crispatus LCR04 (DSM 33487), Lactiplantibacillus plantarum (formerly Lactobacillus plantarum) LP01 (LMG P-21021), Lactiplantibacillus plantarum (formerly Lactobacillus plantarum) LP02 (LMG P-21020), Lactobacillus salivarius sub. salivarius LS03 (DSM 22776), Lactiplantibacillus plantarum (formerly Lactobacillus plantarum) LP09 (DSM 25710), Lactiplantibacillus plantarum (formerly Lactobacillus plantarum) LP14 (DSM 33401), Bifidobacterium longum BL03 (DSM 16603), Lactobacillus gasseri LGS01 (DSM 18299), and mixtures thereof.

6. The strain of bacteria for use according to claim 1, wherein said strain of bacteria is for use in a treatment method of preventing, or inhibiting, or reducing, a viral infection of the respiratory apparatus by a coronavirus, preferably SARS-CoV-2 virus, in a subject in need thereof, and associated pathologies or symptoms.

7. A mixture for use in a treatment method of preventing, or inhibiting, or reducing a viral infection of the respiratory apparatus in a subject in need thereof, and associated diseases or symptoms, said mixture comprising at least two strains, preferably at least three strains, according to claim 1.

8. The mixture for use according to claim 7, wherein said mixture comprises Lactobacillus crispatus LCR01 (DSM 24619), Lactobacillus crispatus LCR04 (DSM 33487), Limosilactobacillus fermentum (formerly Lactobacillus fermentum) LF15 (DSM 26955), Lactiplantibacillus plantarum (formerly Lactobacillus plantarum) LP01 (LMG P-21021), Lactiplantibacillus plantarum (formerly Lactobacillus plantarum) LP02 (LMG P-21020), Lactobacillus salivarius sub. salivarius LS03 (DSM 22776), Bifidobacterium bifidum BB10 (DSM 33678), Lactobacillus plantarum LP09 (DSM 25710), Lactiplantibacillus plantarum (formerly Lactobacillus plantarum) LP14 (DSM 33401), Bifidobacterium longum BL03 (DSM 16603), Lactobacillus gasseri LGS01 (DSM 18299), and mixtures thereof.

9. The mixture for use according to claim 7, wherein said mixture further comprises at least one prebiotic ingredient, or prebiotic fibre; preferably said at least one prebiotic ingredient, or prebiotic fibre, is selected from the group comprising or, alternatively, consisting of inulin, fructo-oligosaccharide (FOS), galacto-oligosaccharide (GOS), guar gum and mixtures thereof, preferably inulin.

10. A composition for use in a treatment method of preventing, or inhibiting, a viral respiratory infection in a subject, and associated diseases or symptoms, comprising:
a strain of bacteria according to claim 1; and
an additive or excipient of food grade or pharmaceutically acceptable grade, preferably said composition is for use in a treatment method of preventing, or inhibiting, or curing a viral infection of the respiratory apparatus by a coronavirus, preferably SARS-CoV-2 virus, in a subject, and associated diseases or symptoms.

11. A method of treatment comprising:
administering to a subject that has a respiratory virus a bacteria selected from the group consisting of:
(a) Lactobacillus brevis LBR01 (DSM 23034);
(b) Lactobacillus crispatus LCR01 (DSM 24619), Lactobacillus crispatus LCR04 (DSM 33487) or a mixture thereof;
(c) Limosilactobacillus fermentum (formerly Lactobacillus fermentum) LF10 (DSM19187), Limosilactobacillus fermentum (formerly Lactobacillus fermentum) LF15 (DSM 26955) or a mixture thereof;
(d) Lactobacillus gasseri LGS01(DSM 18299);
(e) Lactiplantibacillus plantarum LP01 (LMG P-21021) Lactiplantibacillus plantarum, LP02 (LMG P-21020), Lactiplantibacillus plantarum LP09 (DSM 25710), Lactiplantibacillus plantarum LP14 (DSM 33401) or a mixture thereof;
(f) Limosilactobacillus reuteri LRE02 (DSM 23878), Limosilactobacillus reuteri LRE03 (DSM 23879), Limosilactobacillus reuteri LRE11 (DSM 33827), or a mixture thereof;
(g) Lactobacillus salivarius sub. salivarius LS03 (DSM 22776);
(h) Bifidobacterium adolescentis BA02 (EI-15) (DSM 18351);
(i) Bifidobacterium bifidum BB01 (DSM 22892), Bifidobacterium bifidum BB10 (DSM 33678), or a mixture thereof;
(j) Bifidobacterium breve BR03 (DSM 16604), Bifidobacterium breve B632 (DSM 24706), or a mixture thereof;
(k) Bifidobacterium infantis B102 (DSM 24687);
(l) Bifidobacterium lactis BS01 (LMG P-21384); and
(m) Bifidobacterium longum BL03 (DSM 16603), Bifidobacterium longum BL04 (DSM23233), Bifidobacterium longum DLBL07 (DSM 25669), Bifidobacterium longum DLBL08 (DSM 25670), Bifidobacterium longum DLBL 10 (DSM 25672), Bifidobacterium longum DLBL 11 (DSM 25673), or a mixture thereof or a supernatant thereof.

12. The method of claim 11, wherein said bacteria is formulated as a probiotic.

13. The method of claim 11, wherein said bacteria is selected from the group consisting of: Lactobacillus crispatus LCR01 (DSM 24619), Lactobacillus crispatus LCR04 (DSM 33487), Limosilactobacillus fermentum (formerly Lactobacillus fermentum) LF15 (DSM 26955), Lactiplantibacillus plantarum (formerly Lactobacillus plantarum) LP01 (LMG P-21021), Lactiplantibacillus plantarum (formerly Lactobacillus plantarum) LP02 (LMG P-21020), Lactobacillus salivarius sub. salivarius LS03 (DSM 22776), Bifidobacterium bifidum BB10 (DSM 33678), and mixtures thereof.

14. The method of claim 11, wherein the subject has a coronavirus infection.

15. The method of claim 11, wherein the subject has a SARS-CoV-2 virus infection.
